(19) United States
(12) Patent Application Publication
(10) Pub. No.: US 2008/0052110 A1
(43) Pub. Date: Feb. 28, 2008

(54) Referral system

(75) Inventors: Joseph A. Ruggirello, Macomb Township, MI (US); Jack Koyle, Waterford, MI (US)

Correspondence Address: HARNESS, DICKEY & PIERCE, P.L.C., P.O. BOX 828, BLOOMFIELD HILLS, MI, 48303, US

(73) Assignee: Cambridge Software Solutions Corp., Waterford, MI (US)

(21) Appl. No.: 11/508,398

(22) Filed: Aug. 23, 2006

(51) Int. Cl. G06Q 10/00; A61B 5/00

(52) U.S. Cl. 705/2; 600/300

(57) ABSTRACT

A referral service system includes a user interface receiving, at a service location of a service provider, information identifying a referred entity. The service provider is a specialist or general practitioner in a professional field or trade that involves routinely rendering non-referral related services to persons seeking those services, and routinely evaluating the persons to determine need for referral to another service provider. A referral record output module generates a referral record and electronically communicates the referral record to a referee, wherein the referral record includes at least information identifying a referrer, the information identifying the referred entity, and information identifying the referee. A referee location indication module generates an indication of a location of the referee at which the referred entity can obtain services provided by the referee, and communicates the indication of the location of the referee to the referred entity.

### FIELD

The present disclosure generally relates to a referral system, and relates in particular to methods and systems for referring recipients of services from one service provider to another.

### BACKGROUND

The statements in this section merely provide background information related to the present disclosure and may not constitute prior art.

Present referral procedures of referral based business have not changed much over the last fifty years or so. Referees (e.g., doctors, healthcare professionals, businesses, institutions, and/or contractors, etc.), who rely on referrals, have lost information, money, and business due to a gap in transferring information from a referrer (e.g., doctors, healthcare professionals, businesses, institutions, and/or contractors, etc.) to the referee. This gap has also created a lack of follow up for many referrers. Until recently, the vast majority of referrals have occurred by a written referral system. This written referral system relies on the referred entity (e.g., patient, customer, and/or client, etc.) to follow thru by acting on the written referral, which requires that the referred entity not lose any of the written information. The problem with this written based system is that a lot of people can misplace the referee's information. A large percentage of business can be lost this way. Once the pathway is broken, the referred entity may seek professional services elsewhere. In this case, the referee never has any idea that someone was seeking their professional services. Since the referee has no idea that someone was referred to them, there is no way for the referee to contact the referred entity in order to schedule the provision of services (e.g., an appointment, interview, consultation, etc.). This problem also limits the ways for the referred entity to retrieve information on the referee.

### SUMMARY

A referral service system includes a user interface receiving, at a service location of a service provider, information identifying a referred entity. The service provider is a specialist or general practitioner in a professional field or trade that involves routinely rendering non-referral related services to persons seeking those services, and routinely evaluating the persons to determine need for referral to another service provider. A referral record output module generates a referral record and electronically communicates the referral record to a referee, wherein the referral record includes at least information identifying a referrer, the information identifying the referred entity, and information identifying the referee. A referee location indication module generates an indication of a location of the referee at which the referred entity can obtain services provided by the referee, and communicates the indication of the location of the referee to the referred entity.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a block diagram illustrating a referral service system.

FIG. 2 is a flow diagram illustrating a method of inputting data identifying a referred entity into a user interface of the referral service system of FIG. 1.

FIG. 3 is a flow diagram illustrating a method of inputting referrer identifying data into a user interface of the referral service system of FIG. 1.

FIG. 4 is a flow diagram illustrating a method of entering data identifying required services into a user interface of the referral service system of FIG. 1, including symptom(s) and/or problem(s) and need for referral exhibited by the referred entity.

FIG. 5 is a flow diagram illustrating a method of entering required services identifying data into a user interface of the referral service system of FIG. 1, including reason for the referral and/or specific problems to be evaluated by the referee.

FIG. 6 is a flow diagram illustrating a method of entering service location identifying data into a user interface of the referral service system of FIG. 1, including one or more specific locations that the referred entity has selected, such as an address of the referee.

FIG. 7 is a flow diagram illustrating a method of generating output data executed by output function(s) of the referral system of FIG. 1, including printing and/or e-mail functions.

FIG. 8 is a block diagram illustrating a referral service system utilizing stand alone referral software running on a referrer's office equipment.

FIG. 9 is a graphical view of a user interface of the referral service system.

FIG. 10 is a flow diagram illustrating a method of providing referral services for use with the referral service system of FIG. 9.

FIG. 11 is a block diagram illustrating a network distributed referral service system.

FIG. 12 is a functional block diagram illustrating functional components of the network distributed referral service system of FIG. 11.

FIG. 13 is a graphical view of a user interface of the network distributed referral service system of FIGS. 11 and 12.

FIG. 14 is a method of providing referral services for use with the network distributed referral service system of FIGS. 11-13.

FIG. 15 is a block diagram illustrating a peer to peer referral service system.

FIG. 16 is a block diagram illustrating a method of providing referral services for use with the peer to peer referral service system of FIG. 15.

### DETAILED DESCRIPTION

The following description is merely exemplary in nature and is not intended to limit the present disclosure, application, or uses.

By way of overview, the referral service system includes a user interface receiving, at a service location of a first service provider, information identifying a referred entity. The service provider is a specialist or general practitioner in a professional field or trade that involves routinely rendering non-referral related services to persons seeking those services. Also, in the course of or adjunct to providing the non-referral related services, practice in the professional field or trade involves routinely evaluating the persons to determine whether the recipients are in need of referrals to one or more second service providers for additional services of another type, such as those provided by a general practitioner or specialist in a different field or trade, or in a related field or trade.

The service provided by the first service provider is one that the persons tend to seek out for its own sake; thus, it is not simply a referral service. For example, the first service provider can be a general dentist, while the second service provider can be an orthodontist. Also, the first service provider can be an Attorney of one specialty, while the second service provider can be an Attorney of another specialty. Further, the first service provider can be a real estate agent, while the second service provider can be a real estate broker. Thus, persons seeking out the first service provider do not typically do so solely for the purpose of obtaining a referral to the second service provider.

A referral record output module generates a referral record, such as a referral letter, and electronically communicates the referral record to a referee. The referral record includes at least information identifying a referrer, the information identifying the referred entity, and information identifying the referee. A referee location indication module generates an indication of a location of the referee at which the referred entity can obtain services provided by the referee, and communicates the indication of the location of the referee to the referred entity. As noted above, the referral record is communicated to the referee electronically, such as by email, text message, and/or facsimile transmission. In some embodiments, the referral record can be communicated electronically directly from the referrer to the referee. In additional or alternative embodiments, the referral record can be sent to the referee indirectly, such as by sending the referral record to a website service that maintains an inbox for the referee and/or follows up with referred entities on behalf of the referee. In some embodiments, such a website can also expect to receive confirmation from the referee and/or referred entity that an appointment has been made. In additional or alternative embodiments, such a website can generate an electronic reminder to the referrer, the referee, and/or the referred entity after a given amount of time has passed without the appointment being made or kept.

In some embodiments, such as those adapted to needs of the medical profession, the referrer and referee are both medical professionals, and the referral record is a referral letter that fulfills the needs of referred entities for such a referral letter in order to receive treatment by the referee in accordance with requirements by their insurance carriers. In this case, the referral record that is electronically communicated is fundamentally different in form and legal significance from the type of referral that can be sent by a mere referral service, which cannot provide the type of referral needed in such a circumstance.

The referral service system can be provided in the form of software. In some embodiments, the software can be given to a referred entity by a referee in the course of establishing a professional relationship. In alternative or additional embodiments, the software can be downloaded from a website by the referee or the referred entity. In still additional or alternative embodiments, the software can be adapted to the needs of different service industries and particular referees who are service providers within those service industries. Herein, the term “service” is broadened to including making, use, or sale of products.

In some embodiments, the referral service system addresses some of the aforementioned problems by providing an improved system and apparatus, including a computer software program. The software program can assist in solving the problems associated with hand written referrals between referred entities and referees.

In additional or alternative embodiments, the referral service system addresses some of the aforementioned problems by providing a method of referral in which a referred entity is provided with detailed driving instructions and/or a map directing the referred entity to one or more locations of the referee. This mapping/driving service can be performed via Internet, website, and/or a preloaded map.

In still additional or alternative embodiments, the referral service system addresses some of the aforementioned problems by including information, such as a customized header, for the referee's location. Such a header can include, for example, a proper title, pictures, biography, or anything else deemed necessary for the referee.

In yet additional or alternative embodiments, the referral service system addresses some of the aforementioned problems by including contact information of the referrers. The general information can include, for example, name, address, town, city, zip code, phone number, fax, email and/or web site.

In still additional or alternative embodiments, the referral service system addresses some of the aforementioned problems by use of a preloaded map of the referee's location, which can optionally be supplemented with additional information, including, for example, address, town, city, zip code, phone number, fax, email, and/or web site.

In yet additional or alternative embodiments, the referral service system addresses some of the aforementioned problems by providing the referees with the ability to track new referrals of referred entities who have not made contact or are unable to make contact with the referees.

In still additional or alternative embodiments, the referral service system addresses some of the aforementioned problems by empowering referrers and referees to utilize email and/or mail to communicate information identifying needed services, such as procedure specific and non-specific information data, to the referred entities before and after their appointments.

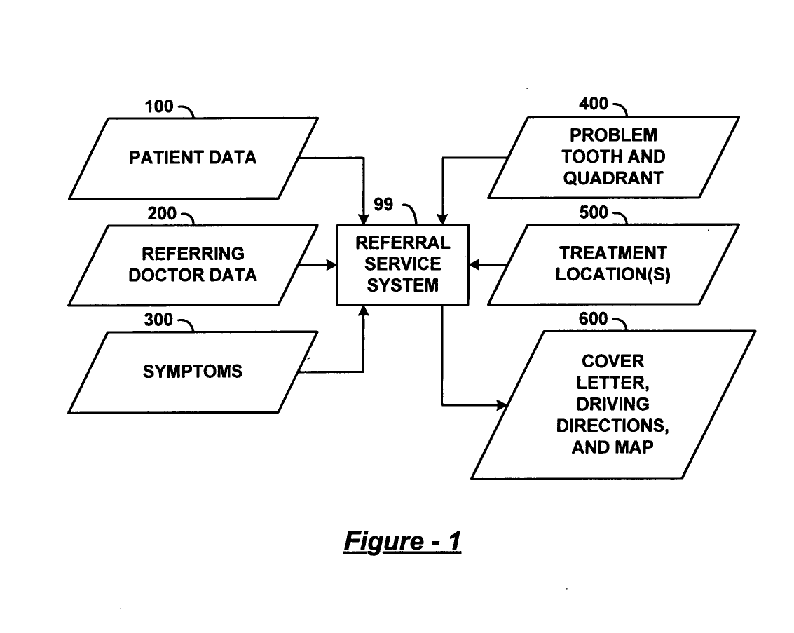

Turning now to FIG. 1, an embodiment of the referral service system 99 is particular described that takes the form of software to be run on a general practitioner dentist's office computer in order to refer a patient to a particular specialist dentist (e.g., orthodontist, periodontist, etc.). However, it should be readily understood that additional or alternative embodiments can accomplish referrals in alternative ways and/or other contexts. For example, additional or alternative embodiments can refer clients from one health professional to another, from one lawyer to another, from real estate agents to mortgage brokers, from interior designers to contractors, etc.

In the particular embodiment illustrated in FIG. 1, the system 99 receives data identifying the referee by method 100 in the form of patient identifying data. The system 99 also receives referrer identifying data by method 200 in the form of data identifying the general practitioner. Further, the system 99 receives data identifying required services by methods 300 and 400 in the form of symptoms and problem tooth and quadrant. The system 99 still further receives data identifying a referrer by receiving a selection identifying a service location by method 500 in the form of an office of the particular specialist at which the patient is to receive treatment. Finally, the system 99 generates output data by method 600 in the form of an introduction, cover letter, driving instructions and map, and e-mails.

It should be readily understood that the system 99 can be implemented on a stand alone PC. It is not important whether the PC is networked, although Internet access is required for e-mail functions to work. In some embodiments, email functions are either optional or not employed; thus, Internet access is not required. Yet, in some embodiments having Internet access, a user of system 99 can be notified via the Internet and/or the program updated automatically if any modifications are required (e.g., an office is added, some text needs to be changed, etc.).

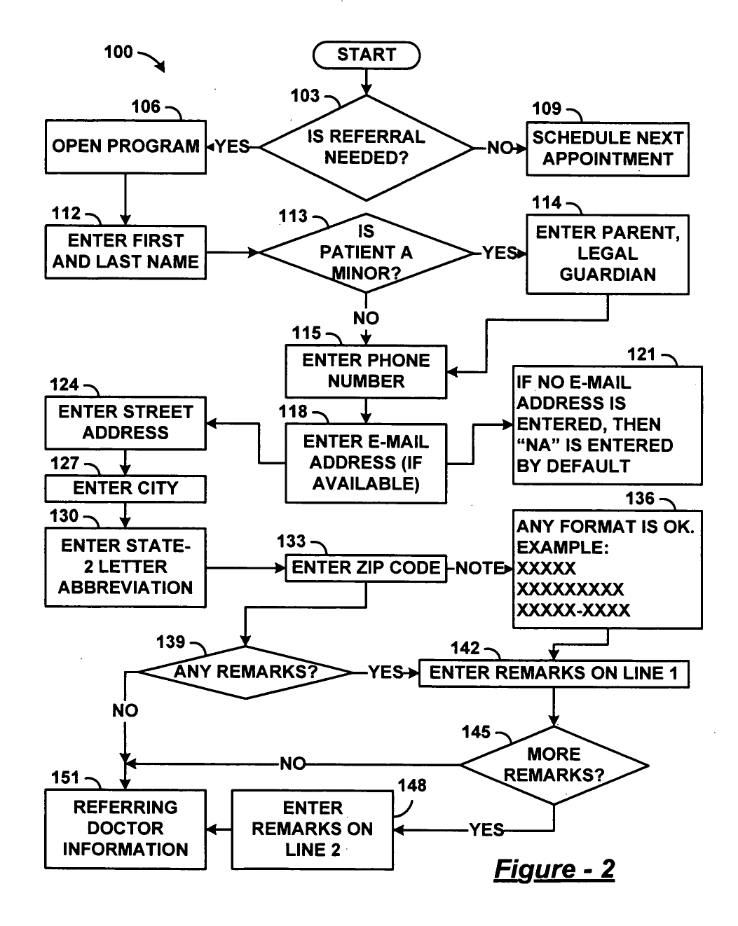

Turning now to FIG. 2, method 100 accomplishes data entry for data identifying the referrer. Method 100 first determines if a referral is needed at step 103. If no referral is needed, the next appointment, if necessary, is scheduled at step 109. If a referral is needed, the user starts the program at step 106. In some embodiments, all of the information identifying the referred entity can be imported from the referrer's pre-existing database. Alternatively or additionally, information can be entered manually.

Manual entry of the information identifying the referred entity can start, for example, with entry of the patient's first and last name at step 112. If the patient is a minor at decision step 113, then the name of the parent or legal guardian is entered at step 114. Next, a phone number is entered for the referred entity at step 115, plus an email address at step 118, if available. If there is no e-mail address, then “NA” can be entered automatically at step 121. The referred entity's street address is entered at step 124, plus the city at step 127, the state at step 130 (e.g., 2 letter abbreviation), and the zip code at step 133, for which multiple formats can be accepted and processed at step 136. Example multiple formats include: 5-digit xxxxx, 9-digit xxxxxxxxx, or 10-digit xxxxxxxxxx. Sometimes, additional remarks may be necessary. If so at decision step 139, a number of characters (e.g., up to 80 characters) can be entered on one line at step 142. If more characters are necessary at decision step 145, then more characters can be entered on a second line at step 148. Finally, at step 151, method 100 ends by proceeding to method 200 (FIG. 1).

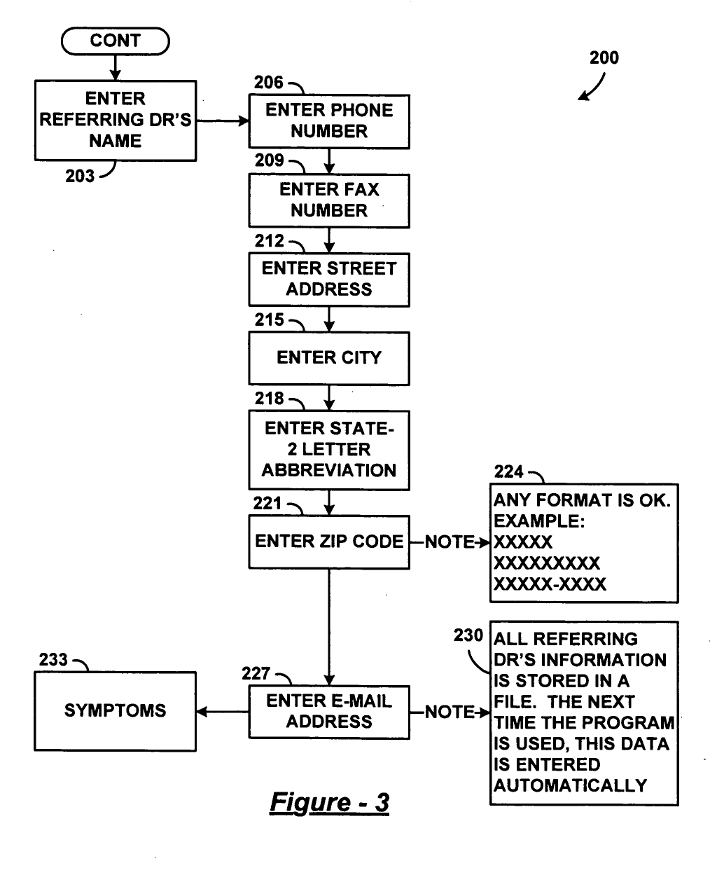

Turning now to FIG. 3, method 200 accomplishes entry of data identifying the referrer, including entry of the referring doctor's name at step 203, phone number at step 206, fax number at step 209, street address at step 212, city at step 215, state at step 218 (e.g., two letter abbreviation), and zip code at step 221. Again, any zip code format can be accepted at step 224. Example multiple formats include: 5-digit xxxxx, 9 digit xxxxxxxxx, or 10-digit xxxxxxxxxx. The referrer's email e-mail address can also be entered at step 227. All of this information can be stored in a file at step 230 so that the next time this person makes a referral, this information can be entered automatically. Finally, method 200 ends at step 233 by proceeding to method 300 (FIG. 1).

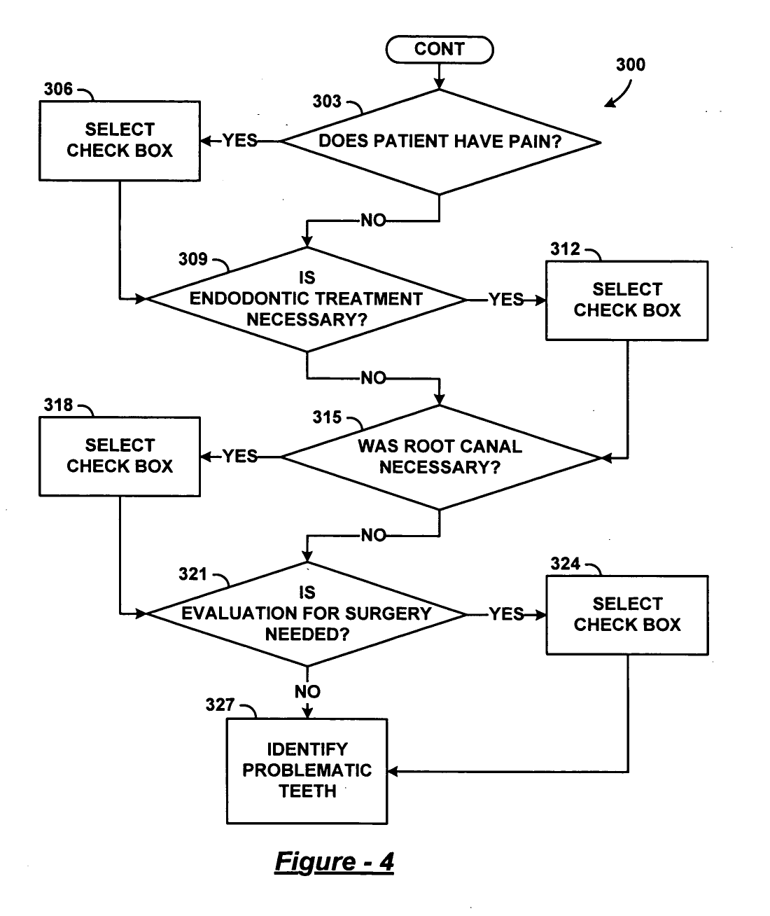

Turning now to FIG. 4, method 300 accomplishes entry of some of the data the helps the dental specialist trouble shoot the problem of rendering services to the patient. There can be, for example, a number of selection decisions that are presented to the user, including: whether there is presence of pain at selection decision step 303; whether endodontic treatment is necessary at selection decision step 309; whether root canal treatment was started at selection decision step 315; and whether evaluation for surgery is needed at selection decision step 321. It is envisioned that more or less selection decisions can be presented, and that an initial set of presented decisions can be made modifiable and/or expandable to suit the needs of a particular specialist. For example, and initial set of choices, such as four choices, can be expanded up to as many choices as needed. Each selection decision step is followed by a step 306, 312, 318, and 324 of selecting or not selecting a check box or other user interface component interaction to indicate the user selection. Finally, method 300 ends at step 327 by proceeding to method 400 (FIG. 1).

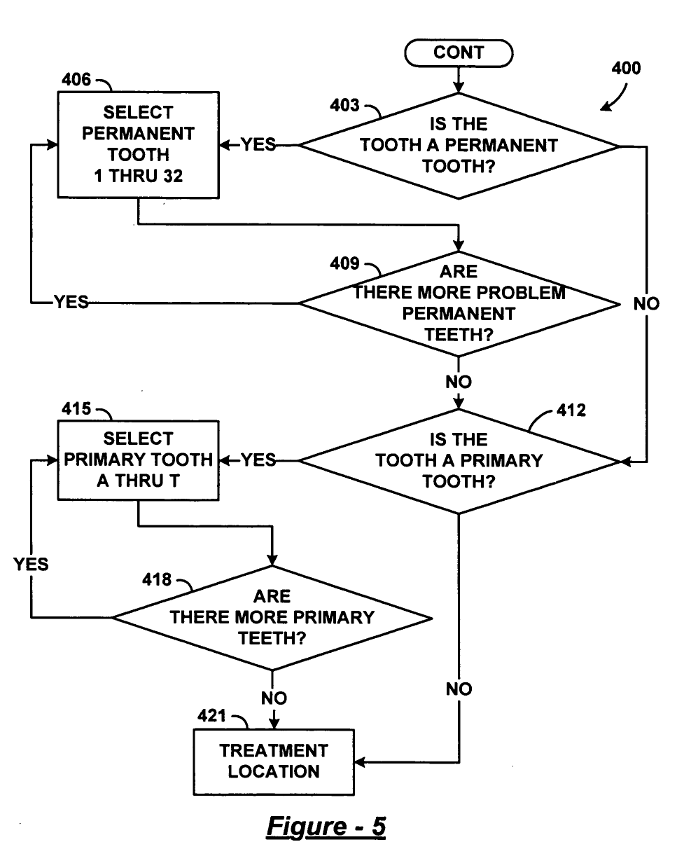

Turning now to FIG. 5, method 400, accomplishes entry of some more data the helps the dental specialist trouble shoot the problem of rendering services to the patient. For example, this data entry can identify one or more unique tooth numbers and/or quadrants that is/are the source of the problem. There can be, for example, an additional number of selection decisions that are presented to the user, including whether the problem tooth is a permanent tooth at selection decision step 403. If so, the user selects a permanent tooth at 406 among 32 possibilities. Then, if there are more problem permanent teeth at decision step 409, then the user selects more of the possible permanent teeth at step 406. Alternatively or additionally, the user can be required to select whether there is a problem tooth that is a primary tooth. If so, the user selects one of twenty possible teeth at step 415. Then, if there are more problem primary teeth at decision step 418, then the user selects more of the possible primary teeth at step 415. Again, it is envisioned that more or less selection decisions can be presented, and that an initial set of presented decisions can be made modifiable and/or expandable to suit the needs of a particular specialist. For example, an initial set of choices can be expanded to as many as needed. Finally, method 400 ends at step 421 by proceeding to method 500 (FIG. 1).

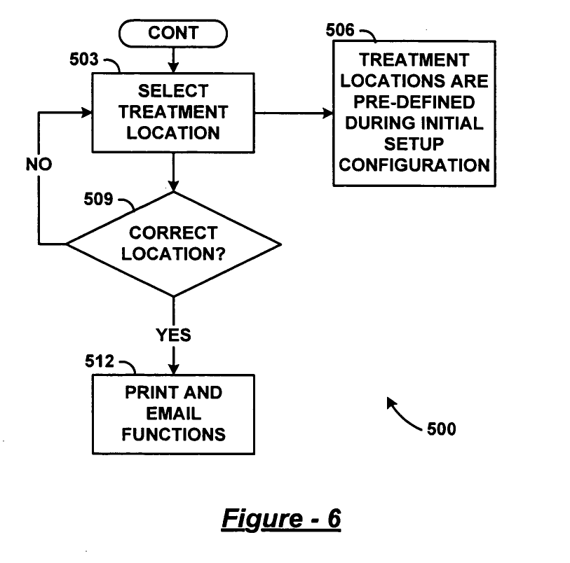

Turning now to FIG. 6, method 500 allows the referred entity to choose at step 503 a location at which to receive dental treatment services rendered by the specialist. The locations can be pre-defined at step 506 during the initial setup configuration. In some embodiments, the system software program can allow as many locations to be defined as needed. The user of the software selects the location on behalf of the referred entity at selection decision step 509. It should be readily understood that if there is only one referee with only one location predefined, then the data identifying the referee is received during predefinition of the location, and the user selection to open the dedicated program serves to select that referee and that location. It is envisioned that the referrer can have a folder or folders filled with icons for opening different, dedicated referral software programs. Finally, method 500 ends at step 512 by proceeding to method 600 (FIG. 1).

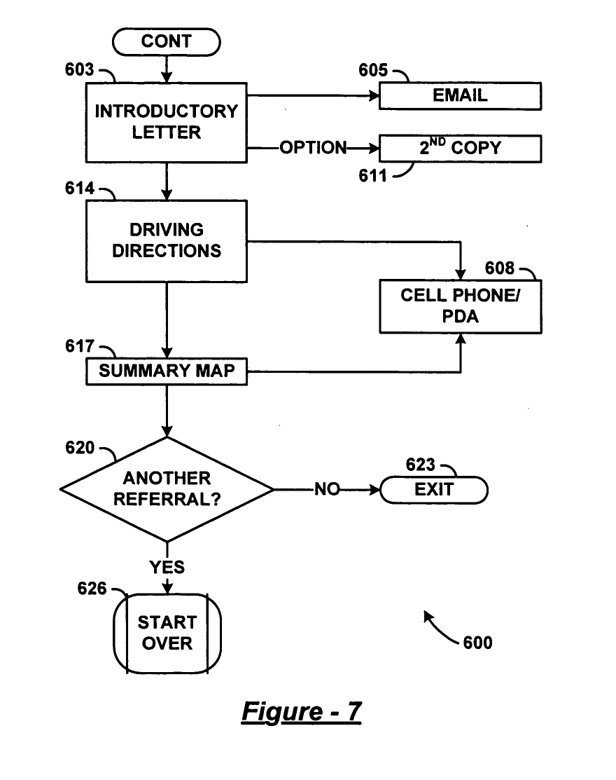

Turning now to FIG. 7 method 600 accomplishes various software output functions, which generate various documents electronically for printing, fax, and/or email. For example, an introduction/cover letter can be generated at step 603 and automatically emailed at step 605 to the dental specialist's inbox, with a copy also being sent to an email inbox of a provider of the software. This referral record can additionally or alternatively be printed in a format for mailing to the referee dentist specialist. This letter introduces the referred entity to the specialist. The letter contains a header that describes the specialist. In some embodiments, it also has a body including six paragraphs. The contents of the six paragraphs are defined here:

### Paragraph 1: Person's Name that is Being Introduced

Address

City, State, Zip

Phone

e-mail address (if available)

### Paragraph 2: Person's Name of Business Making the Referral

Address

City, State, Zip

Phone Number, Fax Number

E-mail address (if available)

### Paragraph 3: Remarks Section

Line one—maximum of 80 characters

Line two—maximum of 80 characters

### Paragraph 4: Symptoms

This area is pre-defined during the initial setup configuration. This can be expanded to as many choices as needed for an additional fee.

### Paragraph 5: Problem Tooth/Area

There can be a maximum number of choices, such as thirty-two choices.

### Paragraph 6: Treatment Center Location

Name of the location

Street Number and Name

Suite

City, State, Zip

Phone

E-mail address (if available)

If there is no e-mail address for the specialist, a copy of the introductory letter can be e-mailed to the patient's email inbox, the parent or legal guardian's inbox, a website of the software provider, and/or the referrer. There is also an option to print a second copy, if needed, at step 611, for mailing, placement in the patient's file, etc. The system also generates driving instructions at step 614 that show turn-by-turn driving instructions from the referred entity's address to the specialist's address. The system additionally generates a map at step 617 that shows an overview of the FROM address and the TO address. The driving instructions and map can be printed and additionally sent automatically to the referred entity's cell phone or PDA by email, text message, etc. at step 608. In alternative or additional embodiments, a text message reminder can be sent to the referred entity's cell phone and/or the introductory letter can be copied to the referred entity's email inbox. Also, a text message can be sent to the cell phone/PDA (608) of the Patient, Business Health Care Professional or Institution who has a cell phone or PDA and wants to be reminded. In some embodiments, merely the name and address of the referee can be sent by email or text message to the an electronic inbox of the referred entity. Alternatively or additionally, a hyperlink can accompany the address that allows the referred entity to automatically access a mapping/routing service and use the address to obtain a map and/or directions. If another referral is needed at step 620, then the user can start over at step 626; otherwise the program exits at step 623.

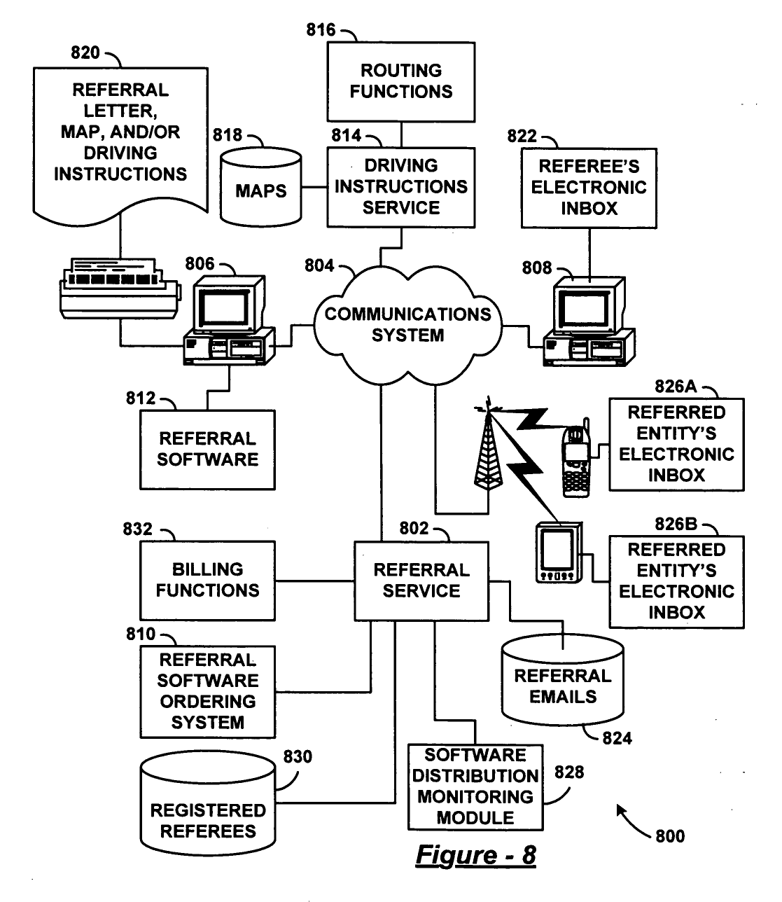

Turning now to FIG. 8, the referral service system 800 described above can be partially web based, with a referral service 802 of the software provider, such as a website, connected to a referrer's office computer 806 and a referee's office computer 808 by a communications system 804, such as the Internet. For example, the referee can access referral service 802 and use referral software ordering system 810 to provide information for customizing referral software 812 to the needs of the referee. The software provider can then customize the software and license a number of copies to the referee, which can then distribute those copies to referrers.

The referee can later access ordering system 810 to expand the license, and receive additional or alternative copies of the software from the software provider. The referee can also utilize ordering system 810 to request changes to the referral software 812, resulting in automatic updating of the software 812 running on computer 806 by service 802. Referral software 812 can access a driving instructions service 814 over communications system 814. Software 812 can call methods of service 814 and provide appropriate arguments for service 814 to utilize maps 818 and routing functions 816 in order to produce the map and driving instructions. Software 812 can print the referral record, map, and driving instructions at the referrer's office at 820. Software 812 can also send copies of any or all of that information to an email or text message inbox 826A and 826B of the referred entity. Software 812 can additionally send copies of the referral emails to the referee's email inbox 822, and to the referral service for storage in datastore 824. In some embodiments, the copy stored in datastore 824 can be abridged to lack the data identifying the referred entity or describing the problem to be solved. However, the copy stored in datastore 824 at least identifies the referrer and the referee. Software distribution monitoring module 828 then periodically accesses datastore 824 and a datastore 828 of registered referee's and their licensing information, and compare the number of referrers identified in datastore 824 for that referee against that referee's licensing allowance in datastore 830. If the referee's licensing allowance is exceeded, then module 828 alerts referral service 802 that the referee has evidently made and distributed illegal copies of the software 812. In some embodiments, module 802 can automatically disable some or all copies of software 812 that are distributed by the referee. In some embodiments, the referral record can be communicated electronically, and especially by email, to a referral system software provider. In additional or alternative embodiments, the referral system software can be configured to send a copy of the referral record first to the software provider and disable viewing, printing, and any additional electronic transmission of the referral record until a return email is received from the software provider in response to the email. Alternatively or additionally, each copy of the referral system software can require that it be registered electronically with the software provider.

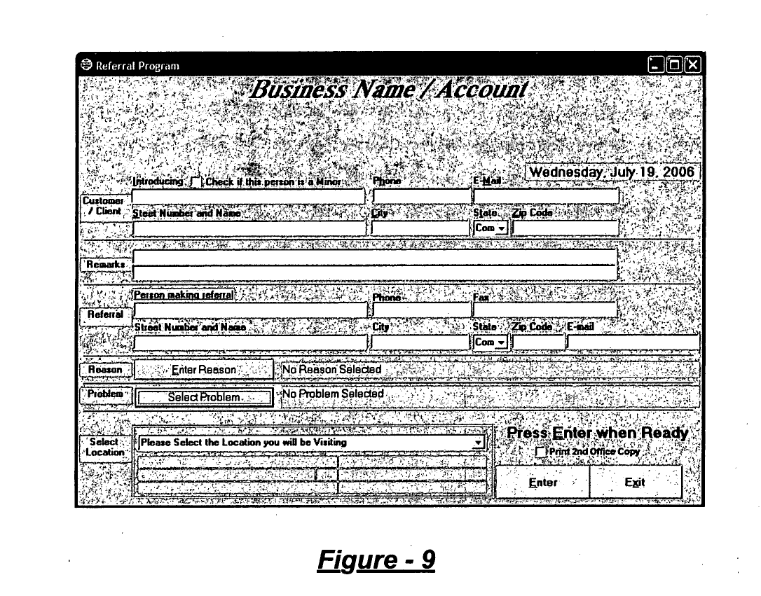

Turning now to FIG. 9, a user interface of the referral software can be designed to elicit the needed data for methods 100-500 (FIG. 1). It should be readily understood that the user interface components for eliciting the data can be arranged on the interface in order to elicit the information according to the sequence detailed above. Yet, the user can enter the information in order the user desires.

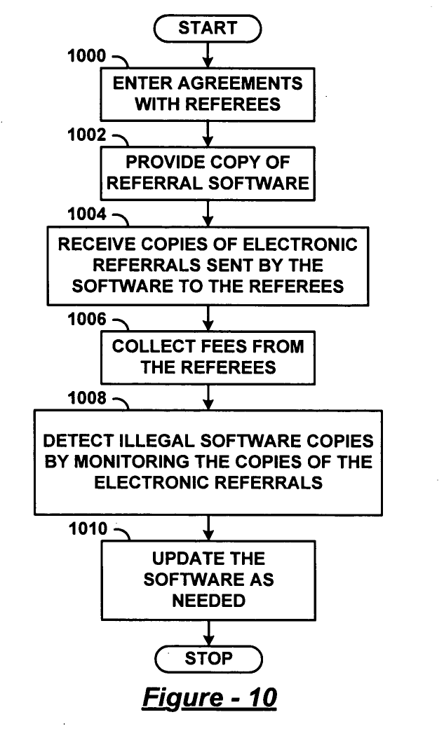

Turning now to FIG. 10, the method of providing referral services can be utilized with the embodiment(s) described above. It should be readily understood that this same method can be used with versions of the software adapted for various contexts. Beginning at step 1000, agreements are entered with the referees. For example, a referee can agree to license a number of copies of the software, and the software provider can agree to customize a version of the software for that referee and/or provide copies of the referral software at step 1002 for the referee to distribute to referrers. The agreement can also entail requirements that all parties initiating and/or receiving referrals are HIPPA compliant.

Customizing the software to the referee can involve entering all of the information identifying the referee and each referee location. In some embodiments, customizing the software can also involve including user interface components designed to elicit information identifying the types of services needed by the referred entity, and these types of components can be varied depending on the profession or trade of the referee. In additional or alternative embodiments, customizing the software can further include adapting the software to include advertisements in referral letters, maps, driving directions, and the like that are targeted to needs of referred entities who are in need of the type of services provided by the referee. Such advertising space can be sold by category so that the software provider to generate additional revenue. Advertisements can be included in some software but not others depending on the category of referee, and/or advertisements can be selected at time materials are generated for the referred entity based on the reasons for referral. In still additional or alternative embodiments, customization can involve write protecting the information identifying the referee, so that it cannot be altered, and the copy cannot be modified by an end user to fabricate a referral to a different referee or additional referees. Similar write protection can protect advertisements.

Then, at step 1004, the software provider can receive copies of referral emails sent by the distributed copies of the software. At any time, the software provider can collect fees from the referees at step 1006, such as an initial per copy fee and/or monthly subscription fee. Meanwhile, the software provider can detect illegal copies of the software at step 1008 by monitoring the copies of the referral emails. Further, at step 1010, the software provider can update copies of the software electronically over a network, such as the Internet, which can result in disablement of illegal copies. Updating the software at step 1010 can alternatively or additionally include receiving requests from referees to alter referee identifying information and/or user interface components and responding by updating the software electronically over the network, or by sending new hard copies of the software to the referee or to referrers identified by the copies of the electronic referrals.

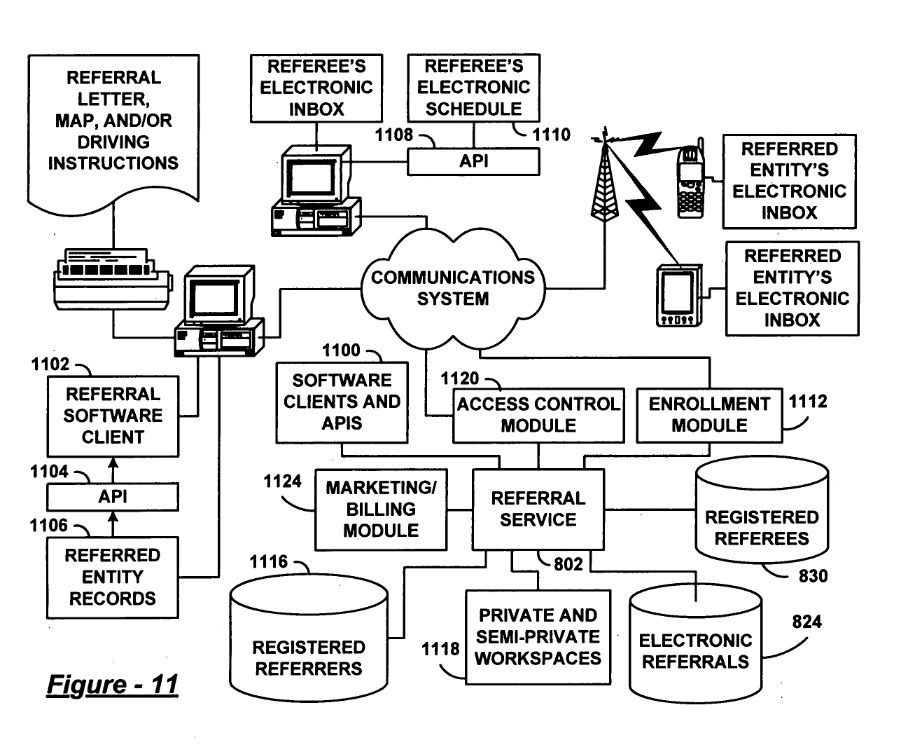

Turning now to FIG. 11, in alternative or additional embodiments, the referral service can be similar to that described above with reference to FIG. 8, but with some significant differences or additions. For example, the software can be provided in the form of software clients with application program interfaces (APIs) 1100. The referrer can download a client 1102 and API 1104 to an office machine at the referrer's location over, for example, the Internet. The API 1104 can allow the referrer to browse and import patient identifying data from referred entity records 1106 already stored on the referrer's office machine. Also, the referee can download an API 1108 that interfaces with the referee's electronic schedule 1110. If the referee does not have an application for an electronic schedule, then the referee can download from service 802 an electronic schedule application.

Referrers and referees can obtain access to the aforementioned resources by accessing enrollment module 1112 to set up an account as either or both of a referrer and a referee. The account information is stored in referee datastore 830 and/or referrer datastore 1116, depending on the type of account. Referrers and referees are also allowed access to their own private and semi-private workspaces 1100 with varying access privileges.

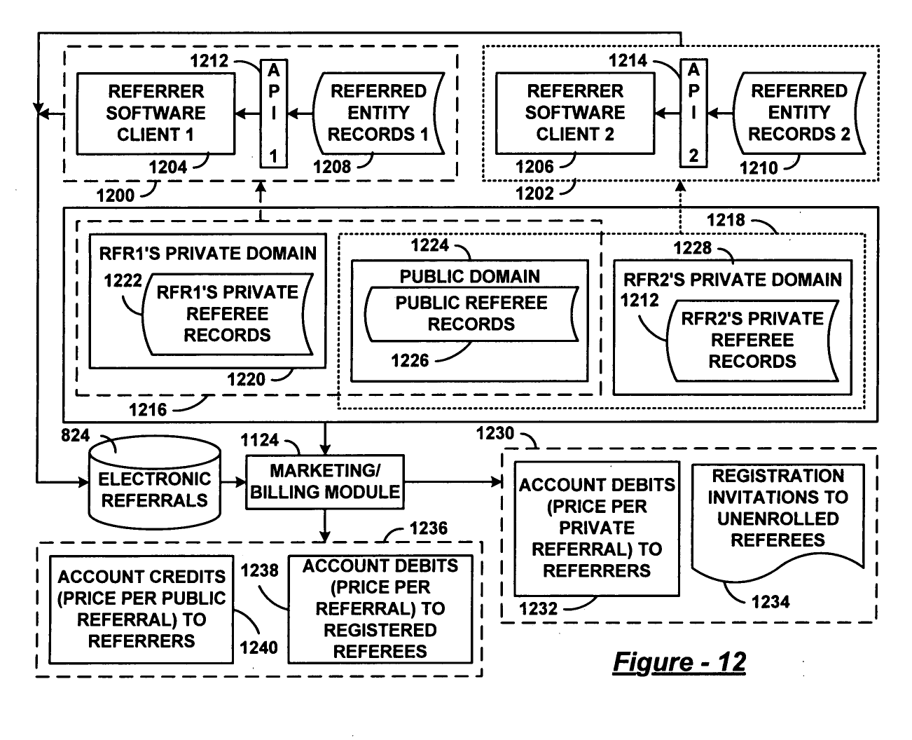

Access to workspaces 1118 and other resources, such as software clients and APIs 1100, can be controlled by access control module 1120. For example, and with particular reference to FIG. 12, two different referrers 1200 and 1202 running separate software clients 1204 and 1206 accessing separate referred entity records 1208 and 1210 by separate APIs 1212 and 1214 can access their own private workspaces 1216 and 1218. Each referrer 1200 and 1202 can access a public domain 1224 that provides information 1226 identifying enrolled specialists. Also, referrer 1200 can access its private domain 1220 in which it stores information 1222 identifying unenrolled specialists known to the referee 1200, but domain 1220 is not accessible to referrer 1202. Meanwhile, referrer 1202 can access its private domain 1228 in which it stores information 1229 identifying unenrolled specialists known to the referee 1202, but domain 1228 is not accessible to referrer 1200.

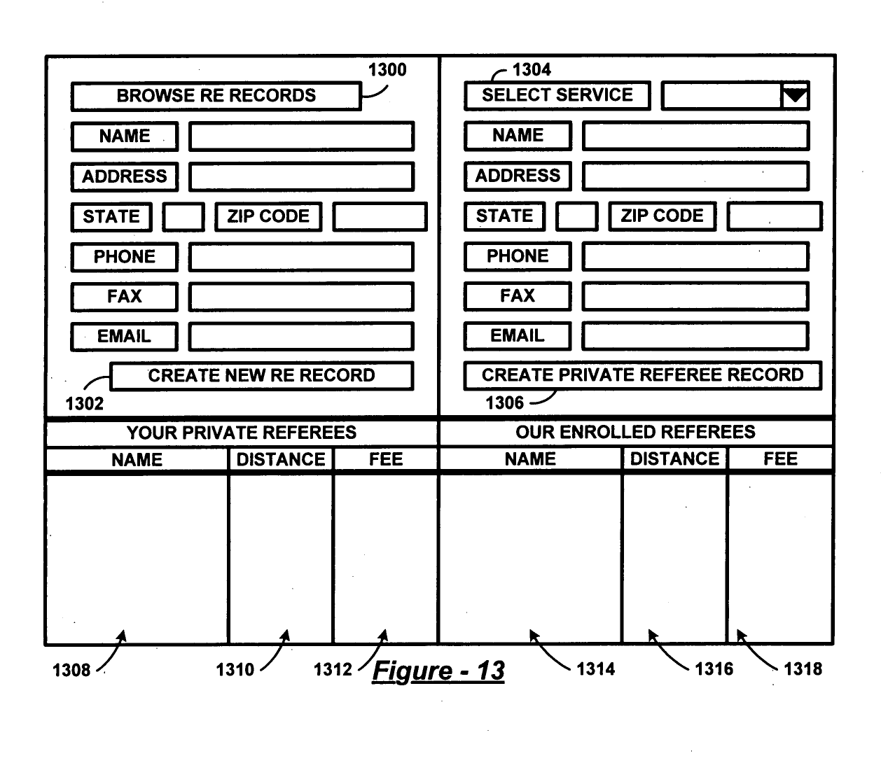

Turning to FIG. 13, a user interface of the software client can have a control 1300 allowing the referrer to browse referred entity records stored in the referrer's record keeping system, and import identifying data. Another control 1302 can allow the referrer, having manually entered the identifying data, to create a new referred entity record in the referrer's record keeping system via the referrer's API. Still another control 1304 can allow the referrer to select a type of service (e.g., medical specialty, legal specialty, etc.), and obtain two lists of referees providing that service. For example, a list of that referrer's private referees for that service is provided that is filterable by name 1308, distance 1310 of the referee's nearest location from the referred entity, and the referral fee 1312 paid by the referee to the referrer for referrals. Also, another list of public referees is provided that is also filterable by name 1314. Also, another list of public referees is provided that is also filterable by name 1314, distance 1316, and referral fee 1318. The information for populating the lists is obtained from the referrer's online workspace, and the referrer can add a private specialist to the private domain of the workspace by manually entering the information and activating another control 1306.

Returning to FIG. 11, workspaces of referees can be used for automated scheduling via API 1108, which synchronizes a schedule 1110 with a copy of the schedule contents if the online referee workspace of workspaces 1118. In this case, access control module 1120 can allow a referred entity who has received an email referral to access the online copy of the referee's schedule via a link in the email referral. A one time use code in the email referral can allow that referred entity to automatically schedule an appointment, and receive in response an automated email with the link and another one time use code allowing rescheduling of their appointment. In operation, available appointment times can be viewed on a calendar, and the referred entity can submit a request for an appointment time. Submission of the request can result in the appointment time being marked as requested in the electronic calendar. This request can be reviewed and confirmed by personnel at the referee's location. The confirmation can result in the appointment time being marked unavailable in the electronic calendar, and a confirmation email being sent the referred entity with the new link and new one time use code. A reschedule request can allow the referred entity to select whether to make their previously scheduled appointment time immediately available, or available contingent upon confirmation of a newly requested appointment time. Each time that the referred entity reschedules an appointment, a new link and one time use code can be sent to the referred entity.

It should be readily understood that personnel at the referrer location, in the presence of the referred entity and interacting therewith, can access the referee's online schedule and at least initially request an appointment time for the referred entity. In operation, the referrer using the software client can access the referee's online electronic schedule before sending a referral email and view the available appointment time. Then, the referral email and the request for the appointment time can be sent simultaneously. Therefore, a user of the software client can have access privileges regarding a referee's workspace at least for scheduling an initial appointment on behalf of a referred entity, without a need for a one time pass code. As a practical matter, the user interface can, upon selection of an enrolled referee, activate certain controls. One of the controls can allow printing of a map and/or directions so the referred entity can assess the route and decide whether the referee is suitable in that respect. Another activated can control allowing the referrer to access that referee's online electronic schedule so the referred entity can assess whether the referee is suitable in terms of availability. This control can alternatively or additionally allow the referrer to access the referee's online electronic schedule with appointment request privileges. Request of an available appointment time can then cause the referral email to be sent, and confirmation of the appointment time can result in the confirmation email being sent to the referred entity. If the appointment time cannot be confirmed, it is envisioned that the referee can telephone or otherwise contact the referred entity in order to reschedule the appointment. The referrer can also be notified of problems automatically so that the referrer can follow up and, if needed, place the referred entity with another referee. Replacement of a referee can result in a recall of the copy of the referral email and the copy sent to the referral service.

Referring generally now to FIGS. 11 and 12, the referral service can also have a marketing/billing module 1124. Module 1124 can access copies of the referral emails stored in datastore 824 in order to perform automated billing. Module 1124 can alternatively or additionally access information about unenrolled specialists in referrers' private domains 1220 and 1228 in order to perform advertising. For example, referral emails sent to enrolled referees can be processed to obtain results 1236, including per referral charges 1238 assessed to enrolled referees. In some embodiments, the referral service 802 can collect referral fees on behalf of the referrers. Therefore, the per referral charges 1238 can be the entire referral fees due to the referrers. In this case, module 1124 can produce, for example, a monthly bill that collects the referral fees from the referees. Then, module 1124 can subtract a service fee, and send the remainder to the referrers, resulting in per referral account credits 1240 to the referrers. If the referrers and referees are enrolled in automatic electronic debit and credit, then the charges 1238 and credits 1240 can be performed electronically either periodically or in real time. In some embodiments, if a referral email is recalled, appropriate adjustments can be made to account balances of referrers and/or referees.

On the other hand, referral emails sent to private referees that are not enrolled can be processed differently to obtain results 1230. For example, the referrer can be responsible for per referral charges 1232, in which case these charges 1232 can be assessed to the referrers. Alternatively or additionally, the information in the referral emails 824 sent to unenrolled referees can be used to generate advertising materials, such as emails, to be sent to these unenrolled referees. The advertising emails can contain a link to the enrollment module 1112 and a one time use code allowing an unenrolled referee to obtain a free trial of the referral services.

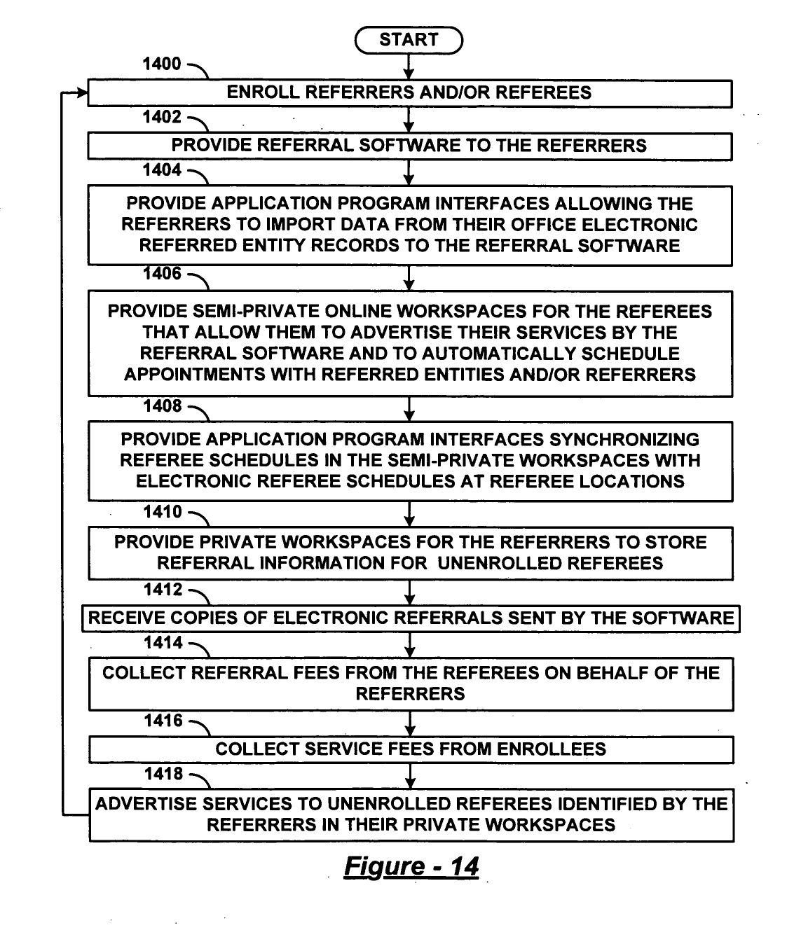

Turning now to FIG. 14, a method of providing referral services according to the embodiments described above with respect to FIGS. 11-13 begins by enrolling referrers and/or referees at step 1400. It is envisioned that the embodiments described above with respect to FIG. 10 can be employed to initially penetrate the marketplace and enroll some referees at step 1400. The embodiments described above with respect to FIG. 10 also supply some referral software, which can be replaced or upgraded to a software client at step 1402, in which the referral software is provided to the referrers. Application program interfaces can also be provided at step 1404 to the referrers that allow them to import data from their office electronic referred entity records to the referral software. At step 1406, private or semi-private online workspaces can be provided to the referees that allow the referee to advertise their services by the referral software, and to automatically schedule appointments with referrers and/or referred entities. In some embodiments, similar workspaces can be provided for advertisers to provide advertisements to be included in materials sent to the referred entities, such as the referral records, maps, driving directions, etc. These advertisements can be uploaded to copies of the software for inclusion in printed materials handed to the referred entity at the referrer's service location, and conditions can be specified for which categories of referred entities and/or referee's initiate inclusion of an advertisement in materials. Thus ad space can be sold by category, and software can be conditionally updated and/or ads conditionally included in materials. In addition, at step 1408, application program interfaces can be provided to the referees that synchronize the referees' electronic schedules running on their office equipment with their electronic schedules in their online workspaces. Further, at step 1410, private or semi-private online workspaces can be provided to the referrers that allow the referrers to store information identifying unenrolled referees that the referrers utilize.

With the referral software clients installed on referrer's office equipment, and with online workspaces provided to referrers and referees, referral emails can be sent to the enrolled and unenrolled referees by the software and copied to the online referral service, resulting in receipt of the copies of the referral emails at step 1412. These copies can be processed to collect referral fees from enrolled referees on behalf of referrers at step 1414, and to collect service fees from the referrers and/or referees at step 1416. Referral services can also be advertised to unenrolled referees identified by referrers in their private workspaces at step 1418, leading to enrollment of additional referees at step 1400.

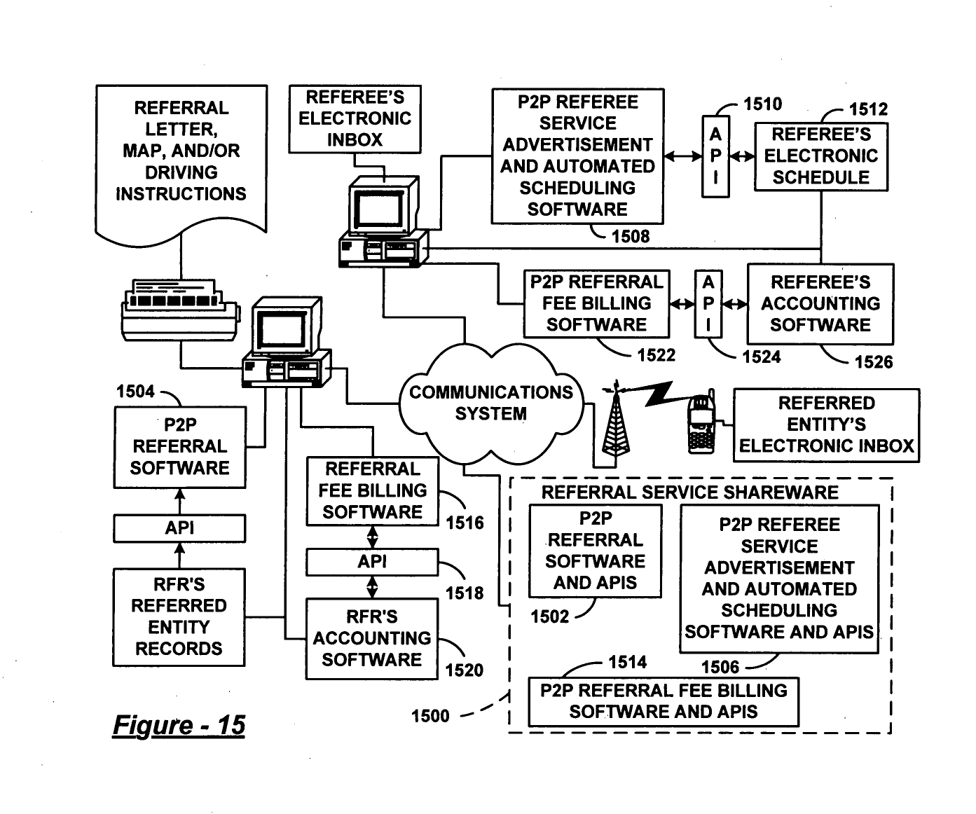

Turning next to FIG. 15, an alternative or additional embodiment of the referral service is similar to the embodiments described above, but with some alterations and/or additions. In particular, resources 1500 of the referral service can be provided in the form of shareware, with referral software and APIs 1502, referee service advertising and automated scheduling software and APIs 1506, and referral fee billing software and APIs being supplied with peer to peer (P2P) functionality. For example, P2P referral software 1504 running on referrer's office equipment can automatically discover referee services advertised by software 1508 running on referees' office equipment. Software 1508 sends out service notifications that specify the referees' types of services, preferred referral record contents, locations, fees, etc. Software 1504 can also call published routines in order to access the referees' electronic schedule 1512 and schedule appointments via software 1508 and API 1510. Each instance of software 1504 can maintain an index of nearby referee services accessible to other instances of software 1504, with the indices being regularly updated by notifications received from software 1508. Software 1508 also has a user interface module for referred entities to access by emailed hyperlinks and call those published routines in order to schedule or reschedule appointments. Additionally, P2P referral software 1504 can allow the referrers to store and access data on referees not listed in the index of that software 1504, and send out advertisement emails to those referees to inform them of availability of the software 1506.

Instances of the referral fee billing software 1520 and 1522 and APIs 1518 and 1524 can run on referrers' and referees' respective office equipment, and interface with accounting software utilized by the referrers and referees. API's 1518 and 1524 allow entry of debits and credits in accounting software 1520 and 1526. Software 1516 and 1522 advertise their services to one another, and automatically process referrals to accomplish automated referral fee bill processing in order to cause bills to be printed and/or accounts to be automatically credited and/or debited.

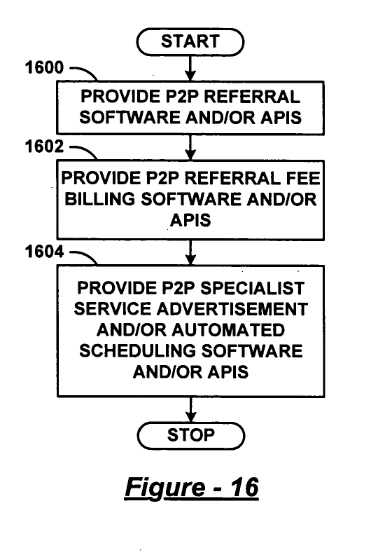

Turning finally to FIG. 16, the method of providing referral services described above with respect to FIG. 15 merely requires provision of P2P software. For example, P2P referral software and APIs can be provided to referrers at step 1600 that discovers and indexes referee services, stores manually entered data for referees not indexed, sends referral emails to referees, schedules appointments with indexed referees, and sends advertisement emails to referees not indexed. Further, P2P referral fee billing software and/or APIs can be provided at step 1602 that prints bills, credits/debits accounts electronically, and/or automatically records credits/debits in accounting software running on referrer and/or referee office equipment. Further, P2P referee service advertisement and automated scheduling software and APIs can be provided at step 1604 that sends notifications of offered services and responds to published routine calls to accomplish automated scheduling of appointments in the referee's electronic schedule.

What is claimed is:

1. A referral service system, comprising:
a user interface receiving, at a service location of a first service provider, information identifying a referred entity, wherein the first service provider is a specialist or general practitioner in a professional field or trade that involves routinely rendering non-referral related services to persons seeking those services, and also, in the course of or adjunct to providing the non-referral related services, routinely evaluating the persons to determine whether the recipients are in need of referrals to one or more second service providers for additional services not provided by the first service provider;
a referral record output module generating a referral record and electronically communicating the referral record to a referee, wherein the referral record includes at least information identifying a referrer, the information identifying the referred entity, and information identifying the referee; and
a referee location indication module generating an indication of a location of the referee at which the referred entity can obtain services provided by the referee, and communicating the indication of the location of the referee to the referred entity.

2. The system of claim 1, wherein said user interface receives data including contact information for contacting the referred entity, and said referral record output module includes the contact information in the referral record.

3. The system of claim 2, wherein said referral record output module communicates the referral record to the referee by utilizing an email address for contacting the referee to email the referral record to an email inbox of the referee.

4. The system of claim 2, wherein said referral record output module communicates the referral record to the referee by printing the referral record in a format for mailing to the referee.

5. The system of claim 1, wherein said referee location indication module electronically communicates the indication of the location of the referee to the referred entity.

6. The system of claim 5, wherein said user interface receives data including an identification of an electronic inbox of the referred entity; and said referee location indication module electronically communicates the indication of the location of the referee to the electronic inbox.

7. The system of claim 6, wherein said user interface receives data including an identification of a cell phone number for contacting the referred entity, and said referee location indication module uses the cell phone number to automatically send the indication of the referee's location by text message to a text message inbox of a cell phone of the referred entity.

8. The system of claim 6, wherein said user interface receives data including an identification of an email address for contacting the referred entity, and said referee location indication module uses the email address to automatically send the indication of the referee's location by email to an email inbox of the referred entity.

9. The system of claim 1, wherein said user interface receives a user selection of an address of the location of the referee.

10. The system of claim 9, wherein said user interface receives data including an identification of an address of the referred entity.

11. The system of claim 10, wherein said referee location indication module generates driving directions navigating a route from the address of the referred entity to the address of the referee.

12. The system of claim 10, wherein said referee location indication module generates a map plotting a route from the address of the referred entity to the address of the referee.

13. The system of claim 9, wherein said referee location indication module generates a map marking the location of the referee.

14. The system of claim 1, wherein said user interface receives data including information identifying services required by the referred entity, and said referral record output module includes the information identifying services required by the referred entity in the referral record.

15. A method of providing referral services, comprising:
entering an agreement with a referee to provide one or more copies of referral system software in exchange for at least one fee, wherein the referral system software is operable to:
(a) receive, at a service location of a first service provider, information identifying a referred entity, wherein the first service provider is a specialist or general practitioner in a professional field or trade that involves routinely rendering non-referral related services to persons seeking those services, and also, in the course of or adjunct to providing the non-referral related services, routinely evaluating the persons to determine whether the recipients are in need of referrals to one or more second service providers for additional services not provided by the first service provider;
(b) generate a referral record and electronically communicate the referral record to the referee, wherein the referral record includes at least information identifying a referrer, the information identifying the referred entity, and information identifying the referee; and
(c) generate an indication of a location of the referee at which the referred entity can obtain services provided by the referee, and communicate the indication of the location of the referee to the referred entity; and
providing one or more copies of the referral system software to the referee for distribution by the referee to referrers.

16. The method of claim 15, further comprising customizing the referral system software according to needs of the referee, including adapting the referral system software to identify the referee and the location of the referee, thereby producing customized referral system software.

17. The method of claim 16, further comprising:
receiving copies of referral emails sent by distributed copies of the customized referral system software; and
detecting illegal copies of the software by monitoring the copies of the referral emails;

18. The method of claim 17, further comprising updating copies of the customized referral system software electronically over a network, including disabling illegal copies of the software.

19. The method of claim 16, further comprising:
receiving a request from the referees to alter the customized referral system software; and
updating the customized referral system software.

20. The method of claim 19, further comprising updating distributed copies of the software electronically over a network.

21. The method of claim 19, further comprising updating by sending new copies of the software to the referee.

22. The method of claim 15, wherein the referral system software is operable to receive data including contact information for contacting the referred entity, and include the contact information in the referral record.

23. The method of claim 15, wherein the referral system software is operable to utilize an email address for contacting the referee to email the referral record to the referee.

24. The method of claim 15, wherein the referral system software is operable to print the referral record in a format for mailing to the referee.

25. The method of claim 15, wherein the referral system software is operable to communicate the indication of the location of the referee to the referred entity.

26. The method of claim 15, wherein the referral system software is operable to receive data including an identification of an electronic inbox of the referred entity, and electronically communicate the indication of the location of the referee to the electronic inbox.

27. The method of claim 26, wherein the referral system software is operable to receive data including information identifying a cell phone number for contacting the referred entity, and use the cell phone number to automatically send the indication of the referee's location by text message to a text message inbox of a cell phone of the referred entity.

28. The method of claim 26, wherein the referral system software is operable to receive data including an email address for contacting the referred entity, and use the email address to automatically send the indication of the referee's location by email to an email inbox of the referred entity.

29. The method of claim 15, wherein the referral system software is operable to receive data including a selection of an address of the location of the referee.

30. The method of claim 29, wherein the referral system software is operable to receive data including an address of the referred entity.

31. The method of claim 30, wherein the referral system software is operable to generate driving directions navigating a route from the address of the referred entity to the address of the referee.

32. The method of claim 30, wherein the referral system software is operable to generate a map plotting a route from the address of the referred entity to the address of the referee.

33. The method of claim 29, wherein the referral system software is operable to generate a map marking the location of the referee.

34. The method of claim 15, wherein the referral system software is operable to receive data including information identifying services required by the referred entity; and include the information identifying the services required by the referred entity in the referral record.
